(19) United States
(12) Patent Application Publication
(10) Pub. No.: US 2007/0219421 A1
(43) Pub. Date: Sep. 20, 2007

(54) Privately Provisioned Survival Supplies Delivery Method

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/464,751

(22) Filed: Aug. 15, 2006

Related U.S. Application Data
(63) Continuation-in-part of application No. 11/462,845, filed on Aug. 7, 2006.
Application No. 11/462,845 is a continuation-in-part of application No. 11/462,795, filed on Aug. 7, 2006.
Application No. 11/462,795 is a continuation-in-part of application No. 11/461,624, filed on Aug. 1, 2006.
Application No. 11/461,624 is a continuation-in-part of application No. 11/461,605, filed on Aug. 1, 2006.
Application No. 11/461,605 is a continuation-in-part of application No. 11/456,472, filed on Jul. 10, 2006.
Application No. 11/456,472 is a continuation-in-part of application No. 11/426,231, filed on Jun. 23, 2006.
Application No. 11/426,231 is a continuation-in-part of application No. 11/425,043, filed on Jun. 19, 2006.
Application No. 11/425,043 is a continuation-in-part of application No. 11/423,594, filed on Jun. 12, 2006.
Application No. 11/423,594 is a continuation-in-part of application No. 11/421,694, filed on Jun. 1, 2006.
Application No. 11/421,694 is a continuation-in-part of application No. 11/420,594, filed on May 26, 2006.
Application No. 11/420,594 is a continuation-in-part of application No. 11/383,022, filed on May 12, 2006.
Application No. 11/383,022 is a continuation-in-part of application No. 11/381,277, filed on May 2, 2006.
Application No. 11/381,277 is a continuation-in-part of application No. 11/381,265, filed on May 2, 2006.
Application No. 11/381,265 is a continuation-in-part of application No. 11/381,257, filed on May 2, 2006.
Application No. 11/381,257 is a continuation-in-part of application No. 11/381,247, filed on May 2, 2006.
Application No. 11/381,247 is a continuation-in-part of application No. 11/379,929, filed on Apr. 24, 2006.
Application No. 11/379,929 is a continuation-in-part of application No. 11/279,333, filed on Apr. 11, 2006.
Application No. 11/279,333 is a continuation-in-part of application No. 11/394,350, filed on Mar. 30, 2006.
Application No. 11/394,350 is a continuation-in-part of application No. 11/384,037, filed on Mar. 17, 2006.

(51) Int. Cl. A61B 5/00

(52) U.S. Cl. 600/300

(57) ABSTRACT

A private civil security subscription mechanism serves to facilitate the provision of survival supplies for corresponding authorized beneficiaries. These supplies may or may not include gender-specific contents that are not specific to a given such beneficiary. These supplies can be retained and delivered as an aggregated unit or can, if desired, be parsed for storage and/or delivery purposes into smaller sub-units. Items as supplied by third parties (including the beneficiary themselves) can also be included with the survival supplies for later delivery. If desired, additional amounts of such supplies than are necessarily required to support such beneficiaries with supplies for a given authorized beneficiary being stored in a plurality of locations. The delivery process will accommodate, if desired, the use of event categorizations to direct the use of differing delivery protocols.

### RELATED APPLICATIONS

This application comprises a continuation-in-part of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265; and

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043; and

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605; and

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

the contents of which are fully incorporated herein by this reference.

### TECHNICAL FIELD

This invention relates generally to providing emergency supplies.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related interactions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swatch of a given civil community. Any number of natural and/or human-caused events can sufficiently disrupt society's infrastructure and ability to provide one or more life-sustaining resources such as water, nutrition, shelter, and the like.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a civilly-catastrophic event occurs.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk survival (let alone the comfort) of their citizens in the face of most civilly-catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

As a result, many (if not most) individuals and their families are largely bereft of the supplies that they will need should a civilly-catastrophic event befall them. This shortcoming tends to be relatively comprehensive; most people have neither a sufficient selection of survival supplies nor a sufficient quantity. For people who do have a store of supplies set aside against such an eventuality, it can be a considerable burden to maintain and ensure the freshness, vitality, and usability of those supplies. At the same time, the same civilly-catastrophic event that occasions their need for supplies will also likely disrupt relevant supply chains enough to cause a partial or complete shortage of supplies at their local merchants. The unfortunate net result is a relatively near term severe need for a variety of survival supplies that will often go unmet for lengthy periods of time.

### BRIEF DESCRIPTION OF THE DRAWINGS

The above needs are at least partially met through provision of the privately provisioned survival supplies delivery method described in the following detailed description, particularly when studied in conjunction with the drawings, wherein:

FIG. 1 comprises a flow diagram as configured in accordance with various embodiments of the invention;

FIG. 2 comprises a flow diagram as configured in accordance with various embodiments of the invention;

FIG. 3 comprises a flow diagram as configured in accordance with various embodiments of the invention;

FIG. 4 comprises a schematic block diagram as configured in accordance with various embodiments of the invention;

FIG. 5 comprises a flow diagram as configured in accordance with various embodiments of the invention;

FIG. 6 comprises a schematic block diagram as configured in accordance with various embodiments of the invention;

FIG. 7 comprises a schematic block diagram as configured in accordance with various embodiments of the invention;

FIG. 8 comprises a flow diagram as configured in accordance with various embodiments of the invention;

FIG. 9 comprises a schematic block diagram as configured in accordance with various embodiments of the invention; and

FIG. 10 comprises a flow diagram as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a subscription-based approach serves to facilitate the procurement of survival supplies for corresponding authorized beneficiaries and the delivery of such supplies to such authorized beneficiaries in response to a civilly-catastrophic event having occurred (and/or, if desired, being imminently likely to occur). Various approaches are set forth with respect to the selection of the survival supplies themselves, their manner of being provided, and/or their delivery

So configured, authorized beneficiaries of such consideration-based private civil security subscriptions will have concrete, predictable access to survival supplies upon the occurrence (and/or threat) of a civilly-catastrophic event. The selection and quantity of emergency survival items can be generally selected (and their maintenance governed) by experts and hence relieve the authorized beneficiary of responsibility in this regard.

These steps are readily facilitated without dependency upon governmental oversight, participation, or control. The particular supplies (and/or the quantity of supplies) provided can vary with the needs and requirements of the authorized beneficiaries. Importantly, via these teachings an individual can take important steps to bring a considerably improved measure of security into their lives, knowing that, should a civilly-catastrophic event indeed be visited upon them, they will have extraordinary and reliable access to survival supplies.

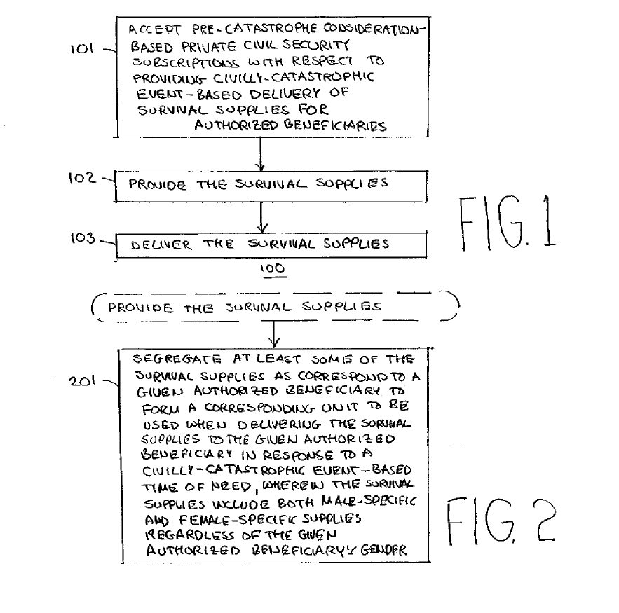

These and other benefits may become clearer upon making a thorough review and study of the following detailed description. Referring now to FIG. 1, an illustrative process 100 provides for accepting 101 consideration-based private civil security subscriptions from corresponding subscribers with respect to providing civilly-catastrophic event-based access to survival supplies. This right of access can pertain, if desired, to a predetermined period of time. For example, a given subscription can relate to providing access to the survival supplies for a one year period of time for one or more authorized beneficiaries as correspond to that subscription.

By one approach, these subscriptions may be accepted by, for example, a for-profit business. By another approach a not-for-profit business (such as a membership-based entity) may be the appropriate entity to offer and accept such subscriptions. As noted, these teachings provide for a subscription-based approach. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extends past the death of a named subscription beneficiary and further allows for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time-based or event-based duration of access rights or, if desired, when a single payment serves to acquire a one-time-only right of access or a perpetual right of access that may be retained, transferred, inherited, or the like);

ownership-based rights of access (as may occur when the subscription provides for ownership rights with respect to the survival supplies);

non-transferable rights of access (as may occur when the subscription, by its terms and conditions, prohibits transfer of the right of access to the survival supplies from a first named beneficiary to another);

transferable rights of access (as may occur when the subscription, by its terms and conditions, permits conditional or unconditional transfer of the right of access from a first named beneficiary to another);

membership-based rights of access (as may occur when the subscription, by its terms and conditions, establishes a membership interest with respect to the accorded right of access such as, for example, a club-based membership);

fractionally-based rights of access (as may occur when the subscription, by its terms and conditions, establishes a divided or undivided co-ownership interest by and between multiple subscription beneficiaries with respect to a right to access the survival supplies); and/or

non-ownership-based rights of access (as may occur when the subscription, by its terms and conditions, establishes the aforementioned right of access via, for example, a lease, rental, or borrowing construct).

If desired, a plurality of differentiated subscription opportunities can be offered in this regard. This plurality of differentiated subscription opportunities can correspond, for example, to providing access to differing selections and/or quantities of survival supplies. As but one very simple illustration in this regard, such subscription opportunities can differ from one another at least with respect to cost. This, in turn, provides subscriber choice with respect to selecting a particular subscription that best meets their specific needs and/or budget limitations.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

As noted, these subscriptions relate to providing access to survival supplies in the event of a civilly-catastrophic event. Such access may be predicated, if desired, upon a requirement that the civilly-catastrophic event be one that persists in substantial form for more than a predetermined period of time (such as one hour, one day, one week, and so forth) or that causes at least a predetermined amount or degree of infrastructure impairment or other measurable impact of choice. In addition, or in lieu thereof, such access may be predicated, if desired, upon a requirement of a particular level of objectively or subjectively ascertained likelihood that a particular category or kind of civilly-catastrophic event will occur within a particular period of time.

As used herein, “civilly-catastrophic event” will be understood to refer to an event that substantially and materially disrupts a society's local, regional, and/or national infrastructure and ability to provide in ordinary course for at least one life-sustaining resource. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation (or worsening) of that interruption.

A civilly-catastrophic event can be occasioned by any of a wide variety of natural and/or non-naturally-caused disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based events (such as collisions with comets, large asteroids, and so forth, extreme solar flares, and the like), extreme environmental events (such as widespread uncontrolled fire or the like), and global or regional pandemics, to note but a few.

Examples of non-naturally-caused disasters capable of initiating a civilly-catastrophic event include both unintended events as well as intentional acts of aggression such as war, terrorism, madness, or the like. Examples of non-naturally-caused disasters capable of such potential scale include, but are not limited to, nuclear-related events (including uncontrolled fission or fusion releases, radiation exposure, and so forth), acts of war, the release of deadly or otherwise disruptive biological or chemical agents or creations, and so forth.

This process 100 then provides for the provision 102 of corresponding survival supplies for these authorized beneficiaries. This can comprise, for example, providing a plurality of life-sustaining resources as pertain to a plurality of differing categories of life's necessities (such as, but not limited to, hydration, nourishment, shelter, clothing, environmentally borne threat abatement, and so forth). Such survival supplies can comprise consumable items (including consumable necessities of human life), non-consumable items (including non-consumable necessities of human life), or both. Illustrative examples of consumable items would include, but are not limited to:

food supplies;

potable water;

batteries;

personal hygiene supplies; and

medical supplies;

to note but a few. Illustrative examples of non-consumable items could include, but are not limited to:

essential clothing items;

bedding items;

food preparation items;

power generation equipment;

repair and maintenance tools;

navigation equipment;

communication items; and

shelter items (such as, for example, a tent);

to again note but a few.

This process 100 will also optionally accommodate providing survival supplies that comprise one or more non-necessities of life. Illustrative examples in this regard might include, but are not limited to:

luxury consumable items;

luxury non-consumable items;

non-essential clothing;

non-human nourishment;

barter medium;

entertainment apparatus;

educational tools and materials;

physical conditioning, exercise, and maintenance training and equipment;

crafts supplies and training; and

sports equipment;

to note but a few.

This step of providing survival supplies can also comprise, if desired, the maintaining of those survival supplies over time to ensure, for example, the freshness, usability, and efficacy of those supplies. Such maintenance can optionally comprise making adjustments to such supplies to reflect dynamically changing circumstances as occur during the consideration-based private civil security subscription period. As one illustration, a new item may become available that is particularly useful in dealing with or otherwise surmounting some condition that may likely arise upon the occurrence of a particular kind of civilly-catastrophic event. In such a case, maintaining such supplies can readily accommodate updating the acquired and stored items to include a supply of this new item.

Accordingly, such maintenance can readily comprise one or more of removing a particular one of the items (as when a better substitute becomes available, when the item itself is shown to be less effective for its intended purpose than was originally thought, and so forth), adding additional ones of a particular item (as when it becomes subsequently understood that more of a particular item is desirable to achieve a particular goal or purpose), adding at least one new stored item that is not already stored (as illustrated in the example provided above), and so forth.

There are numerous optional approaches that may be considered when providing 102 such survival supplies. As a first example in this regard, and referring now to FIG. 2, this provision of survival supplies can further comprise segregating 201 at least some of the survival supplies as correspond to a given one of the authorized beneficiaries to form a corresponding unit to be used when delivering survival supplies to that authorized beneficiary in response to a civilly-catastrophic event, wherein the survival supplies as comprise the corresponding unit include both male-specific and female-specific supplies regardless of the given authorized beneficiary's gender and/or preferences in this regard. Such gender specific supplies are likely, in many application settings, to be in addition to gender-neutral supplies of various kinds and sorts.

By this approach, at least portions of such a unit can be rendered more fungible in that a given unit, while likely including content that is not particularly useful to any given authorized beneficiary, will also likely contain content that is relevant and pertinent to the survival needs of such a person. Such an approach can be particularly beneficial with respect to effecting the proper stocking of inventory, packing, storage, pre-positioning, and delivery of such survival supplies.

The male-specific and female-specific survival supplies can be as varied as desired. By one approach, for example, the male-specific supplies can comprise male-specific toiletries and/or male-specific clothing while the female-specific supplies can comprise female-specific toiletries and/or female-specific clothing. Along these same lines, it would also be possible for this unit to comprise supplies that are at least primarily specific to a relatively wide range of different authorized beneficiary ages ranging, for example, from infancy to elderly. Examples in this regard might include, but are not limited to:

children's clothing;

adult clothing;

children's nutritional supplements;

adult nutritional supplements;

geriatric nutritional supplements;

children's reading material;

adult reading material;

medicines having an adult dosage form factor;

medicines having a child's dosage form factor;

adult entertainment material;

children's entertainment material;

pre-packaged adult-oriented foods; and

pre-packaged child-oriented foods;

to note but a few. Similarly, it would also be possible for such a unit to include size-specific items of clothing for a plurality of differently-sized persons ranging from petite dimensions to very large persons.

As another example regarding the provision of survival supplies, and referring now to FIG. 3, such provisioning can comprise segregating 301 at least some survival supplies as correspond to a given authorized beneficiary to form a corresponding unit (which may or may not include a variety of gender/size/age-specific items as described above). This approach then provides for forming 302 the survival supplies of this unit into a plurality of discrete deliverable sub-units, wherein at least one of the discrete deliverable sub-units comprises a quantity and selection of survival supplies as comprehensively correspond to survival needs of the authorized beneficiary for a particular corresponding period of usage time. (As used herein, “comprehensively” will be understood to refer to a range of survival supplies as will meet a corresponding range of anticipated survival needs as relate to nourishment, hydration, protection from the environment, and so forth.)

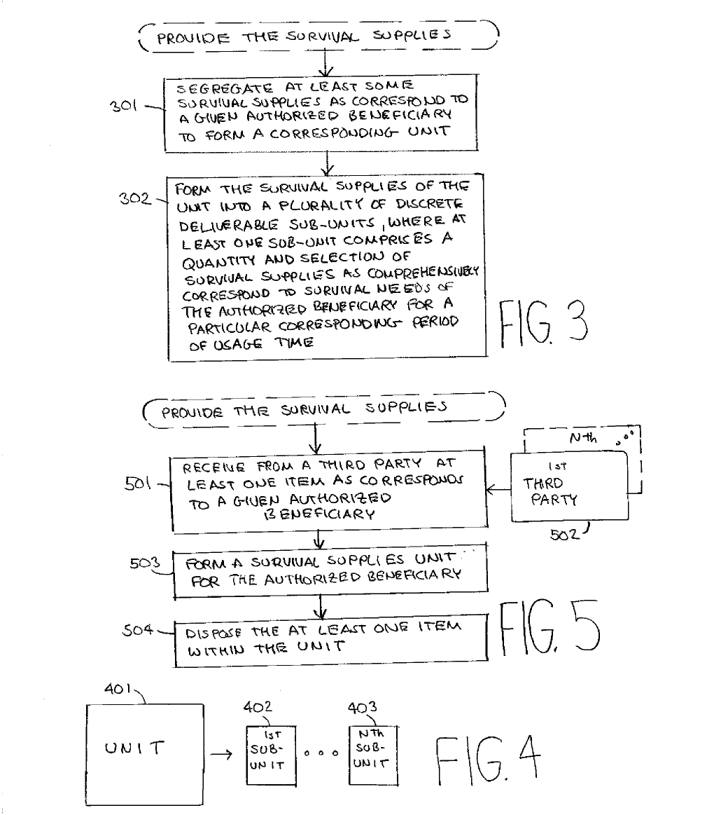

FIG. 4 provides a schematic representation of such a process, where the contents as comprise a unit 401 are parsed amongst a plurality of sub-units (represented here by a first sub-unit 402 through an Nth sub-unit 403 (where “N” will be understood to comprise an integer greater than one)). Those skilled in the art will understand and recognize that such a unit 401 may comprise a physical entity (comprising, for example, a container or room of such supplies) or may comprise a logical entity where the constituent elements of the unit are not necessarily first brought physically together prior to effecting this formation of corresponding sub-units.

As noted above, such a unit 401 may be comprised of consumable survival supplies, non-consumable survival supplies, or a combination of both. It is also possible for these survival supplies to comprise relatively generic supplies and/or one or more items that are specific to a particular given authorized beneficiary. Such specificity may pertain to the authorized beneficiary's gender, age, size, medical condition, religion, cultural background, responsibilities, and/or preferences as the case may be.

By one approach, this unit 401 comprises a portion of the survival supplies as are to correspond to a given authorized beneficiary. By another approach, as desired, this unit 401 comprises substantially all of the survival supplies as correspond to a given authorized beneficiary. These teachings will readily accommodate segregating a sufficient quantity and selection of survival supplies in this manner as will reasonably be required by the authorized beneficiary for some intended and/or identified period of time, such as six months, one year, two years, and so forth.

As noted above, the sub-unit(s) 402 (403) comprise a quantity and selection of survival supplies as will comprehensively correspond to the survival needs of the authorized beneficiary for a particular corresponding period of usage time. Being a lesser collection of items than the unit 401 itself, of course, this period of usage time will be less than the time that is associated with the unit 401. Nevertheless, by one approach, each sub-unit will still provide sufficient provisions for a substantial fraction of the unit's overall capacity in this regard. For example, by one approach, each sub-unit can represent about one fourth of the unit's usage time. In such a case, then, each sub-unit could provide about three month's worth of survival supplies when the unit itself represents about one year's worth of survival supplies.

By one approach, each sub-unit is substantially identical and contains a similar collection of survival supplies. In such a case, for example, two or more of the sub-units may comprise functionally identical non-consumable survival supplies such that these various sub-units, viewed in the aggregate, contain a redundant selection of the functionally identical non-consumable survival supplies. A non-exhaustive listing of examples in this regard might include, but is not limited to, hand tools (such as hatchets, knives, hammers, and so forth), food preparation items (including pots, pans, spatulas, spoons and knives, and so forth), navigational aids (such as compasses, global positioning system platforms, maps, and so forth), fire starting aids (including matches, lighters, flint and striker sets, and so forth), survival instructions (such as how-to guides, routing directions, and so forth), wireless communication devices (including cellular telephones, satellite phones, email and/or Internet-capable platforms, two-way devices such as walkie talkies, and so forth), clothing items, bed clothing (such as sheets, blankets, sleeping bags, inflatable mattresses and pillows, and so forth), and so on.

By another approach, however, differences may be purposefully introduced. For example, a first sub-unit may be half comprised of consumable survival supplies such as food and water while the remaining half comprises tools, shelter materials, communications equipment, and so forth. Second and third sub-units, perhaps intended for later delivery, could be more fully comprised of only consumable survival supplies such as additional food and water. Where the sub-units are similarly sized, it may be that different sub-units in such a case have differing corresponding usage times. For example, the first sub-unit described above might have a usage time of 1.5 months while the second and third sub-units might have a usage time of three months to reflect the quantity of consumable survival supplies that each contains. In such a case, some sub-units might have a substantially shorter corresponding period of usage time than others of the sub-units. Even in such a case, however, at least some of the sub-units will likely represent a significant fraction and portion of the overall usage time as corresponds to the unit itself.

These sub-units 402 and 403 can assume any suitable form factor as befits the needs and/or opportunities as correspond to a given application setting. By one approach at least some of these sub-units may comprise one or more pallets (as are known in the art) that have survival supplies stacked thereon. Various tie-down and/or tarp or other coverings can be employed in such a case to hold the survival supplies in place on the pallet. By another approach, at least some of these sub-units may comprise an open top (or side) or closeable container (made, for example, of wood, metal, plastic, or other suitable material). When closeable, if desired, the container can further be configured as a lockable container. In such a case, the sub-unit could be delivered in a locked state and would be unlocked, presumably, by the intended authorized beneficiary deliveree.

Such sub-units, when employed, can greatly facilitate the storage, distribution, pre-positioning, delivery, and even usage of the aforementioned survival supplies as, for example, three months worth of food represents considerably less volume than one year's worth of food. Such an approach may greatly facilitate ensuring rapid and accurate delivery of such supplies when delivery conditions are likely considerably less than optimum. Such an approach may also facilitate ensuring that such supplies, when delivered, are fresh and will remain so during the period of usage time as corresponds to that sub-unit.

As yet another example of providing survival supplies in accordance with these teachings, and referring now to FIG. 5, if desired this process will accommodate receiving 501 from at least one third party 502 at least one item as corresponds to a given authorized beneficiary. This third party 502 can comprise, for example, the authorized beneficiary themselves, the subscriber, or the like. This third party 502 can also comprise, for example, another party such as a licensed alcoholic beverage sales entity, a licensed tobacco sales entity, a licensed firearms sales entity, a licensed pharmacist, or the like. As to the latter, such items may comprise items of particular interest to a given authorized beneficiary but which, for various reasons relating to regulations, taxing, and so forth, may require initial provisioning via such a third party.

A survival supplies unit as corresponds to this authorized beneficiary can then be formed 503 and this at least one item as received 501 from the third party(s) 502 disposed 504 therein. So configured, when this corresponding unit is delivered to the given authorized beneficiary, the given authorized beneficiary will receive the at least one item along with the survival supplies as comprise that delivered unit.

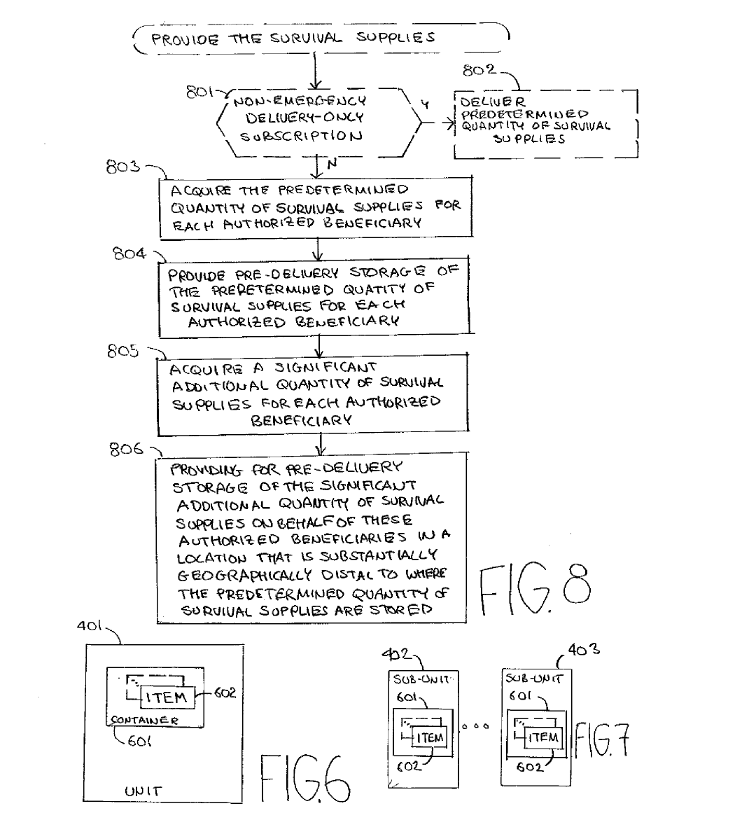

This can comprise, by one approach and referring now to FIG. 6, placing the at least one item 602 within a closeable container 601 that is itself then received within the corresponding unit 401. Such a closeable container 601 may comprise a locked container if desired (to thereby control access to the at least one item) and may have a form factor that is configured and arranged to fit appropriate and suitably within the unit 401. By one approach, if desired, such a container can have one or more windows to thereby permit visual inspection of an interior portion (and presumably of the item(s) itself) of the closeable container.

When the survival supplies as comprise the unit have been segregated into a plurality of sub-units as described above, and referring now to FIG. 7, this item 602 and its container 601 can be similarly placed into the sub-unit 402. In such a case, of course, it would be possible to apportion the at least one item (when such is possible) or to distribute multiple items over a plurality of sub-units as is also illustrated in FIG. 7.

So configured and practiced, personal items and/or items of personal interest to a specific given authorized beneficiary can be readily accommodated. Such items can relate specifically to physical survival needs of the authorized beneficiary or can represent items that cater instead in other ways to the authorized beneficiary. When the item-containing-container comprises a relatively standard container (as viewed by the provider of these services), such a practice also greatly simplifies the task of identifying and moving such an item from one unit (or sub-unit) to another unit (or sub-unit) as may be required during the facilitation of these services.

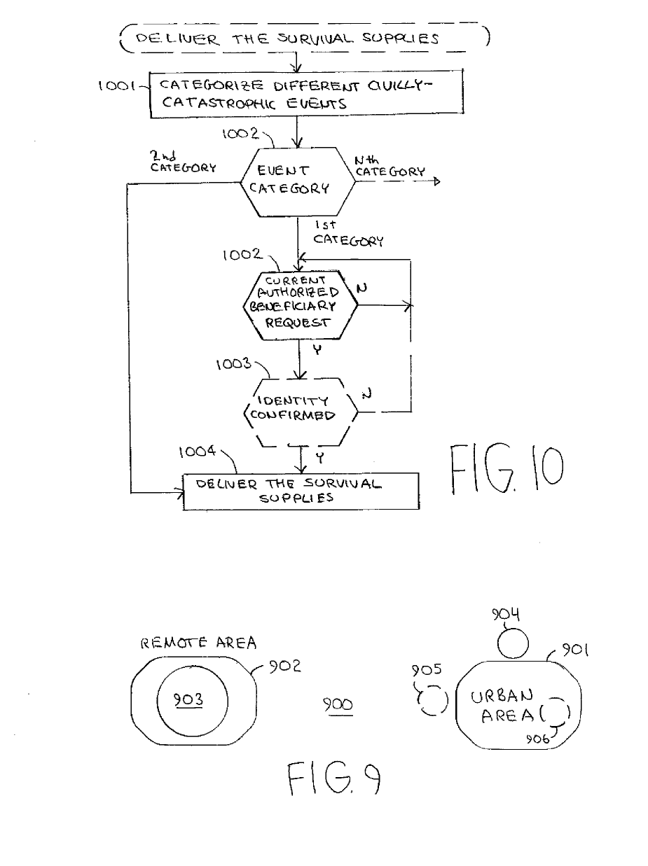

As yet another example of a way by which these survival supplies may be provided, and referring now to FIG. 8, these teachings will accommodate optionally permitting a kind of pre-catastrophe consideration-based private civil security subscription 801 where a given subscriber arranges for the non-emergency pre-civilly-catastrophic event-based delivery of the aforementioned quantity of survival supplies to one or more corresponding authorized beneficiaries. In such a case, these teachings will accommodate delivering such survival supplies to the authorized beneficiary(s) in the absence of any particular triggering catastrophic event.

With continued reference to FIG. 8, if desired, these teachings will also accommodate acquiring 803 the predetermined quantity of survival supplies for each authorized beneficiary (for example, as described above) and then providing for the pre-delivery storage 804 of that predetermined quantity of survival supplies for each such authorized beneficiary. These teachings can then further provide for acquiring 805 a significant additional quantity of survival supplies for each such authorized beneficiary and then also providing for pre-delivery storage 806 of those additional survival supplies on behalf of each authorized beneficiary in a location that is substantially geographically distal to where the predetermined quantity of survival supplies are stored.

This significant additional quantity of survival supplies will typically comprise considerably more than a trivial quantity. By one measure, when the predetermined quantity of survival supplies as are acquired 803 for each authorized beneficiary represents a first period of usage time, the additional quantity of survival supplies will, in turn, represent a significant fraction of that first period of usage time (such as one fourth, one half, or even an equal amount of more). To illustrate, when the predetermined quantity of survival supplies represents about one year of usage time, the additional quantity of survival supplies can represent, for example, about one half year of usage time. (Those skilled in the art will recognize and understand that such examples are intended for illustrative purposes only and are not intended to suggest specific limitations in this regard.)

As noted, these two quantities of survival supplies are stored geographically distal from one another. To illustrate by example (and again without intended to suggest any particular limitations in this regard) and referring now to FIG. 9, the predetermined quantity of survival supplies 903 may be stored in a remote area 902 as compared to an urban area 901 where the authorized beneficiaries as correspond to this store of supplies are typically located. “Remote” is of course a relative term and an appropriate distance in this regard will be governed, in large measure, by a distance that is sufficient to likely protect these supplies from civilly-catastrophic events of concern that may impact the area(s) that typically contain the corresponding authorized beneficiaries. For many such risks, a distance of at least 300 miles is probably prudent though somewhat greater or lesser distances may be necessary or possible in some application settings.

To continue with this example, the supplemental survival supplies 904 are stored considerably closer to the authorized beneficiaries and hence, in this example, to the urban area 901. If desired, these supplemental survival supplies can be themselves further parsed and stored in various closer-in locations as exemplified by the location denoted with reference numeral 905. The specific location of these supplemental survival supplies can vary with the circumstances and may be, for example, on the outskirts of such an urban area 901 as described above or within the urban area as exemplified by the location denoted with reference numeral 906. For many application settings, it may be useful to locate such supplemental supplies within, say, about 75 miles of the corresponding authorized beneficiaries.

It will be understood by those skilled in the art that these supplemental survival supplies are indeed “supplemental.” In particular, by one approach, these supplies are acquired and stored on the effective behalf of the authorized beneficiaries notwithstanding that the subscriptions as pertain to these authorized beneficiaries provide only for the delivery of the predetermined quantity of survival supplies. Accordingly, this significant additional quantity of survival supplies genuinely represents over capacity in this regard. By one approach, this acquisition and storage of supplemental stores in this manner occurs relatively transparently; the authorized beneficiaries may or may not be aware of such provisioning. By another approach, if desired, additional consideration may be required from the subscriber in order to effect such acquisition and storage of supplemental items. In that case, the subscription may explicitly provide for the acquisition and storage of a greater quantity of survival supplies than are actually required to be delivered to the authorized beneficiaries.

So configured, a given authorized beneficiary receives increased comfort with respect to the likelihood that their survival supplies will themselves survive the civilly-catastrophic event that occasions the need for their delivery. In addition, and in some cases perhaps more importantly, such an approach offers delivery options and capabilities that greatly improve a service provider's ability to provide such supplies in a timely and effective manner notwithstanding the tumult, confusion, and chaos that may likely accompany such an event. Further description in this regard appears below.

Referring again to FIG. 1, this process 100 then provides for delivering 103 these survival supplies to these authorized beneficiaries in response to a civilly-catastrophic event (and/or the credible near term threat of such an event). As will be shown below, there are various ways by which such delivery can be accomplished.

For example, when the unit has been parsed into sub-units as described above, such delivery can play out over a period of time. In this case, a first sub-unit may be quickly delivered to provide the authorized beneficiary with immediately-need survival supplies. Additional sub-units can then be delivered as previously delivered supplies wan. Such deliveries may be offered on an automatic basis or in response to a specific authorized beneficiary request.

As another example, when additional survival supplies have been procured as described above, with at least some of the additional supplies being stored relatively closer to the authorized beneficiaries, an initial relatively immediate delivery of survival supplies can be offered from those supplemental stores. The relative proximity of such supplies to the authorized beneficiary can be a powerful offset to a partially or fully inoperable transportation/delivery infrastructure environment. And, if those proximally-located supplies were themselves partially or fully compromised by the effects of the corresponding civilly-catastrophic event, then the more remotely located stores can be drawn upon to provision the authorized beneficiaries as per their subscriptions. In such a case, of course, it is possible that both the primary stock and the supplemental stock of survival supplies as corresponds to given authorized beneficiaries will survive and be available for distribution. If desired, the aggregated surplus can be provided to non-authorized beneficiaries to, in effect, offer aid and comfort to others within the afflicted region.

If desired, delivery can also be conditioned, at least in part, upon the category, kind, and/or severity of the civilly-catastrophic event itself. With continued reference to FIG. 10, these teachings will accommodate categorizing 1001 a plurality of different civilly-catastrophic events to provide a plurality of civilly-catastrophic event categories. This can comprise, for example, categorizing civilly-catastrophic events, at least in part, with respect to their relative temporal impact. That is, a duration of time over when the event and its aftermath are expected to substantially impair society's infrastructure. A very short-lived event, such as an act of aggression that destroys a power generating facility and thereby denies a given region of electricity, may not in and of itself constitute a sufficiently long-lived event to warrant the delivery of survival supplies as per these teachings (presuming that another source of electricity can relatively quickly be brought to bear). On the other hand, a longer-lived event/aftermath may increase the likelihood that such supplies are going to be needed.

This could also comprise, for example, categorizing civilly-catastrophic events, at least in part, with respect to their relative geographic scope of impact. An intense, and even long-lived, event that has only a very small footprint (such as a single house) may not constitute a civilly-catastrophic event whereas an event that is milder, but of considerably great geographic scope (as when the event impacts, in some adverse manner or another, some significant element of society's infrastructure) may represent a greater concern in this regard.

Yet another point to potentially consider is the relative size of a likely-impacted population group. In general, the larger the size of people who are adversely impacted by a given event, the greater the commensurate need and challenge of continuing to provide those people with the necessities of life. Other points of consideration might include, but are not limited to, seasonal considerations, climate and/or weather considerations, as well as the day of the week and/or the time of day, as all of these factors can potentially contribute to mitigating, or aggravating, the impact of a given civilly-catastrophic event and a corresponding need for survival supplies delivery.

By one approach, this step of categorizing civilly-catastrophic events can comprise categorizing such events as being one of two available categories. If desired, however, additional categories can be used to provide, for example, greater resolution with respect to severity and likely need of assistance. For example, if desired, four different escalating categories can be used to characterize a given set of candidate civilly-catastrophic events.

Pursuant to this approach, then, when a given civilly-catastrophic event occurs (and/or when such an event is threatened to occur with a sufficient sense of impending likelihood), a determination 1002 is made regarding the applicable categorization of this particular event. By one approach, when the event corresponds to a first category (such as a category that corresponds to a low tier or mid tier threat), delivery can be conditioned upon receiving 1002 a current request from an authorized beneficiary for the aforementioned survival supplies. This can comprise, for example, a request that is made within no more than a predetermined period of time prior to the occurrence of the civilly-catastrophic event if desired. This can also comprise, in lieu of the latter approach or in combination therewith, a request that is made subsequent to the civilly-catastrophic event having begun.

These teachings will accommodate receiving such a request via any number of means and methods including, but not limited thereto, telephone calls, facsimile transmissions, email or other Internet-based mechanisms, terrestrial and/or satellite-based two-way and/or one-way radio transmissions of various kinds, and so forth. It would also be possible, of course, to use visual signals in settings where such an approach might prove effective.

If desired, this process will optionally provide for confirming 1003 the identify of the authorized beneficiary and/or the authorized status of such a request (via, for example, the use of a predetermined secret such as a password, personal identification number, or the like) before permitting further actions.

Upon receiving such a request under such circumstances, this process then provides for delivering 1004 the corresponding survival supplies. As noted above, this can comprise initially delivering all of the survival supplies to which a given authorized beneficiary is ultimately entitled of only a part of such a quantity and selection. The delivery itself can be to a particular location that the authorized beneficiary identifies when making the aforementioned request or can be to a predetermined location. When making a series of deliveries, this can also comprise making those deliveries to differing locations to track the movement of the authorized beneficiary as may occur in the intervening period of time.

By one approach, the presence of the authorized beneficiary may be required to accept delivery of such survival supplies. By another approach, if desired, such a deliver may be effected regardless of whether that authorized beneficiary is present to accept such a delivery. In such a case, it may be beneficial that at least part of the survival supplies are left in a locked container that the authorized beneficiary is able to access (via a key, code, characterizing biometric, or the like).

In such a case, the delivery of survival supplies is preconditioned upon both a triggering event or circumstance having occurred as well as a specific request from the authorized beneficiary. This is because the triggering event/circumstance itself has been categorized in a manner that prompts this action. Such an approach may be appropriate, for example, when the event is one that may, or may not, result in a particular authorized beneficiary needing their supplies and where a suitable line of communication is likely available to the authorized beneficiary.

Other circumstances can of course occur. When a particular triggering event/circumstance is of sufficient severity, and hence is categorized accordingly (for example, as a “second category” in this illustration provided), these teachings will also accommodate automatically delivering these survival supplies to the authorized beneficiary without also requiring a request as described above. In such a case, a given authorized beneficiary will now be more tangibly assured that their survival supplies will be reliably delivered notwithstanding their present inability, for example, to make a request for those supplies.

It will be appreciated that these teachings provide for a highly flexible yet powerfully effective way by which a modern citizen can greatly improve their likelihood of surviving a civilly-catastrophic event. These teachings are sufficiently flexible so as to accommodate the needs and desires of a wide-ranging set of potential beneficiaries while nevertheless still tending to ensure adequate access to the basic necessities of life. Though training and some related activity may be provided and encouraged, in general the beneficiary receives these benefits without being required to make the commitment of time, energy, and expertise that would ordinarily be associated with attaining such a high level of civil security.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

I claim:

1. A method comprising:
accepting pre-catastrophe consideration-based private civil security subscriptions from subscribers with respect to providing civilly-catastrophic event-based delivery of survival supplies for authorized beneficiaries;
categorizing a plurality of different civilly-catastrophic events to provide a plurality of civilly-catastrophic event categories;
when a civilly-catastrophic event as corresponds to a first one of the civilly-catastrophic event categories occurs, delivering the survival supplies to an authorized beneficiary provided the authorized beneficiary makes a current request for such delivery;
when a civilly-catastrophic event as corresponds to a second one of the civilly-catastrophic event categories occurs, automatically delivering the survival supplies to the authorized beneficiary regardless of whether the authorized beneficiary makes a current request for such delivery.

2. The method of claim 1 wherein accepting pre-catastrophe consideration-based private civil security subscriptions from subscribers comprises accepting the pre-catastrophe consideration-based private civil security subscriptions at a for-profit business.

3. The method of claim 1 wherein the subscriptions comprise at least one of:
time-limited rights of delivery;
event-limited rights of delivery;
inheritable rights of delivery;
rights of delivery predicated upon a series of periodic payments;
rights of delivery predicated upon a one-time payment;
ownership-based rights of delivery;
non-transferable rights of delivery;
transferable rights of delivery;
membership-based rights of delivery;
fractionally-based rights of delivery;
non-ownership-based rights of delivery;
option-based rights of delivery.

4. The method of claim 1 wherein accepting pre-catastrophe consideration-based private civil security subscriptions from subscribers comprises accepting pre-catastrophe consideration-based private civil security subscriptions from subscribers with respect to providing civilly-catastrophic event-based access to a plurality of life-sustaining resources in addition to delivery of the survival supplies.

5. The method of claim 1 wherein accepting pre-catastrophe consideration-based private civil security subscriptions from subscribers with respect to providing civilly-catastrophic event-based delivery of survival supplies comprises accepting pre-catastrophe consideration-based private civil security subscriptions from subscribers with respect to providing civilly-catastrophic event-based delivery of a plurality of life-sustaining resources as pertain to a plurality of differing categories of life's necessities.

6. The method of claim 5 wherein the plurality of differing categories of life's necessities comprise at least one of:
hydration;
nourishment;
medicine;
shelter;
environmentally-borne threat abatement.

7. The method of claim 1 wherein the civilly-catastrophic event comprises an event that substantially disrupts society's infrastructure and ability to provide at least one life-sustaining resource.

8. The method of claim 7 wherein the civilly-catastrophic event comprises at least one of:
a natural disaster;
an unnaturally-caused disaster.

9. The method of claim 8 wherein the natural disaster comprises at least one of:
a severe weather event;
a severe geophysical event;
a severe astronomical event;
a severe disease-based event;
a severe natural shortage of a life-sustaining resource.

10. The method of claim 8 wherein the unnaturally-caused disaster comprises at least one of:
an act of intentional aggression;
an accident that results in public dispersal of a severe environmentally borne danger.

11. The method of claim 1 wherein categorizing a plurality of different civilly-catastrophic events to provide a plurality of civilly-catastrophic event categories comprises categorizing the different civilly-catastrophic events, at least in part, with respect to their relative temporal impact.

12. The method of claim 1 wherein categorizing a plurality of different civilly-catastrophic events to provide a plurality of civilly-catastrophic event categories comprises categorizing the different civilly-catastrophic events, at least in part, with respect to their geographic scope of impact.

13. The method of claim 1 wherein categorizing a plurality of different civilly-catastrophic events to provide a plurality of civilly-catastrophic event categories comprises categorizing the different civilly-catastrophic events, at least in part, with respect to a likely-impacted population group.

14. The method of claim 13 wherein categorizing the different civilly-catastrophic events, at least in part, with respect to a likely-impacted population group further comprises categorizing the different civilly-catastrophic events, at least in part, with respect to a relative size of the likely-impacted population group.

15. The method of claim 1 wherein categorizing a plurality of different civilly-catastrophic events to provide a plurality of civilly-catastrophic event categories comprises categorizing the different civilly-catastrophic events, at least in part, with respect to a corresponding season.

16. The method of claim 1 wherein categorizing a plurality of different civilly-catastrophic events to provide a plurality of civilly-catastrophic event categories comprises categorizing the different civilly-catastrophic events, at least in part, with respect to a corresponding time of day.

17. The method of claim 1 wherein categorizing a plurality of different civilly-catastrophic events to provide a plurality of civilly-catastrophic event categories comprises categorizing the different civilly-catastrophic events, at least in part, with respect to a corresponding weekly day.

18. The method of claim 1 wherein categorizing a plurality of different civilly-catastrophic events to provide a plurality of civilly-catastrophic event categories comprises categorizing the different civilly-catastrophic events, at least in part, with respect to corresponding weather.

19. The method of claim 1 wherein categorizing a plurality of different civilly-catastrophic events to provide a plurality of civilly-catastrophic event categories comprises categorizing the different civilly-catastrophic events with respect to at least four different categories.

20. The method of claim 1 wherein delivering the survival supplies to an authorized beneficiary provided the authorized beneficiary makes a current request for such delivery comprises receiving the current request within no more than a predetermined period of time prior to occurrence of the civilly-catastrophic event.

21. The method of claim 1 wherein delivering the survival supplies to an authorized beneficiary provided the authorized beneficiary makes a current request for such delivery comprises receiving the current request subsequent to the civilly-catastrophic event having begun.

22. The method of claim 1 wherein delivering the survival supplies to an authorized beneficiary provided the authorized beneficiary makes a current request for such delivery comprises receiving the current request from the authorized beneficiary.

23. The method of claim 22 wherein receiving the current request from the authorized beneficiary further comprises confirming an identity of the authorized beneficiary.

24. The method of claim 23 wherein confirming an identity of the authorized beneficiary comprises receiving a predetermined secret from the authorized beneficiary.

25. The method of claim 1 wherein delivering the survival supplies to an authorized beneficiary provided the authorized beneficiary makes a current request for such delivery further comprises delivering the survival supplies to a specific location as is identified by the authorized beneficiary when making the current request.

26. The method of claim 1 wherein delivering the survival supplies to an authorized beneficiary provided the authorized beneficiary makes a current request for such delivery further comprises delivering the survival supplies to a specific location that is determined prior to the civilly-catastrophic event occurring.

27. The method of claim 1 wherein automatically delivering the survival supplies to the authorized beneficiary comprises first at least attempting to deliver the survival supplies to a first specific location as has been previously identified for the authorized beneficiary prior to the civilly-catastrophic event occurring.

28. The method of claim 27 wherein automatically delivering the survival supplies to the authorized beneficiary further comprises, when the authorized beneficiary is not available at the first specific location to receive the survival supplies, automatically at least attempting to deliver the survival supplies to a second specific location as has been previously identified for the authorized beneficiary prior to the civilly-catastrophic event occurring.

29. The method of claim 1 wherein delivering the survival supplies comprises initially delivering all of the survival supplies to which the authorized beneficiary is ultimately entitled.

30. The method of claim 1 wherein delivering the survival supplies comprises initially delivering only a part of the survival supplies to which the authorized beneficiary is ultimately entitled.

31. The method of claim 30 wherein delivering the survival supplies further comprises subsequently delivering an additional part of the survival supplies to which the authorized beneficiary is ultimately entitled to a different location than to where that initial delivery is provided.

32. The method of claim 1 wherein delivering the survival supplies comprises leaving at least a portion of the survival supplies at a predetermined location for the authorized beneficiary that is ultimately entitled to the survival supplies regardless of whether that authorized beneficiary is present to accept delivery.

33. The method of claim 32 wherein leaving at least a portion of the survival supplies at a predetermined location comprises leaving the at least a portion of the survival supplies in a locked container.

34. The method of claim 1 further comprising:
when a civilly-catastrophic event as corresponds to a first one of the civilly-catastrophic event categories threatens, delivering the survival supplies to an authorized beneficiary provided the authorized beneficiary makes a current request for such delivery.

35. The method of claim 34 further comprising:
when a civilly-catastrophic event as corresponds to a second one of the civilly-catastrophic event categories threatens, automatically delivering the survival supplies to the authorized beneficiary regardless of whether the authorized beneficiary makes a current request for such delivery.
